(19) United States
(12) Patent Application Publication
(10) Pub. No.: US 2014/0031664 A1
(43) Pub. Date: Jan. 30, 2014

(54) RADIOGRAPHIC IMAGING DEVICE

(71) Applicants: Hyosig KANG, Weston, FL (US); Chris LIGHTCAP, Davie, FL (US); David BERMAN, Miami, FL (US)

(72) Inventors: Hyosig KANG, Weston, FL (US); Chris LIGHTCAP, Davie, FL (US); David BERMAN, Miami, FL (US)

(73) Assignee: MAKO Surgical Corp.

(21) Appl. No.: 13/562,163

(22) Filed: Jul. 30, 2012

(51) Int. Cl. A61B 6/00

(52) U.S. Cl. 600/407

(57) ABSTRACT

An imaging system includes a radiation source, a detector fixed to the radiation source such that the radiation source and detector form a hand-held imaging device configured to acquire image data, and a navigation system configured to track a pose of the hand-held imaging device.

### BACKGROUND

The present application relates generally to the field of imaging. Specifically, the present application relates to a radiographic imaging device and related systems and methods.

Image guidance is often utilized during minimally invasive surgeries to enable a surgeon to view, via a display screen, portions of a patient's anatomy that are covered by tissue. Typically, a three-dimensional representation of the relevant portion of the patient's anatomy is created preoperatively, and the representation is displayed on a screen during the procedure. The patient's anatomy is tracked by a navigation system during the procedure, and a computer system continuously updates the representation on the screen in correspondence with movement of the patient. Other objects, such as surgical tools, can also be tracked during the procedure. Surgeons are therefore provided with a real-time view as they are manipulating surgical tools within the patient, facilitating safer surgical procedures and more precise results.

In order for the three-dimensional representation of the patient's anatomy to accurately represent the patient's real anatomy as the patient moves during surgery, the patient's anatomy must be registered to the three-dimensional representation. Registration can be accomplished in a variety of ways, including by 2D/3D registration. 2D/3D registration involves using two-dimensional images of the anatomy to register the anatomy to the preoperative three-dimensional representation of the anatomy. One goal of effective image-guided surgical procedures is to quickly and accurately register the patient's anatomy to the preoperative three-dimensional representation.

### SUMMARY

One embodiment of the invention relates to an imaging system including a radiation source and a detector fixed to the radiation source such that the radiation source and detector form a hand-held imaging device. The hand-held imaging device is configured to acquire image data. The imaging system further includes a navigation system configured to track a pose of the hand-held imaging device.

An additional embodiment relates to a hand-held imaging device including a hand-held frame, a radiation source fixed to the frame, and a detector fixed to the frame. The hand-held imaging device is configured to be tracked by a navigation system.

A further embodiment relates to a method for bone registration including providing a three-dimensional representation of an anatomy of a patient; providing a hand-held imaging device having a hand-held frame, a radiation source fixed to the frame, and a detector fixed to the frame; acquiring a two-dimensional image of the anatomy using the imaging device; tracking a pose of the imaging device with a navigation system; and registering the two-dimensional image with the three-dimensional representation.

Alternative exemplary embodiments relate to other features and combinations of features as may be generally recited in the claims.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a perspective view of an imaging system according to an exemplary embodiment.

FIG. 2 is a perspective view of an embodiment of a hand-held imaging device.

FIG. 3 is a perspective view of an additional embodiment of a hand-held imaging device.

FIG. 4 is a perspective view of an embodiment of a hand-held imaging device during imaging.

FIGS. 5A-5B are side views of an embodiment of a hinged hand-held imaging device.

FIGS. 6A-6B are side views of an embodiment of a collapsible hand-held imaging device.

FIG. 7 is a perspective view of an embodiment of a hand-held imaging device during calibration.

FIG. 8 is a view of a display screen during a method of registration.

FIG. 9 is a flow chart illustrating use of a hand-held imaging device for 2D/3D registration according to an exemplary embodiment.

FIG. 10 illustrates an embodiment of a method for registration.

FIG. 11 is a flow chart illustrating use of a hand-held imaging device for displaying a predicted image according to an exemplary embodiment.

FIG. 12 is a flow chart illustrating use of a hand-held imaging device for assessing the position of an implanted component.

FIG. 13 is a flow chart illustrating various additional applications of the hand-held imaging device.

### DETAILED DESCRIPTION

Before turning to the figures, which illustrate the exemplary embodiments in detail, it should be understood that the application is not limited to the details or methodology set forth in the description or illustrated in the figures. It should also be understood that the terminology is for the purpose of description only and should not be regarded as limiting. For example, several illustrations depict a hand-held imaging device imaging a patient's knee, although the imaging device may be used to image any portion of a patient's anatomy (e.g. shoulder, arm, elbow, hands, legs, feet, neck, face, teeth, etc.). Furthermore, in addition to applications related to the medical industry, the imaging device has applications in any industry in which it would be useful to obtain radiographic images.

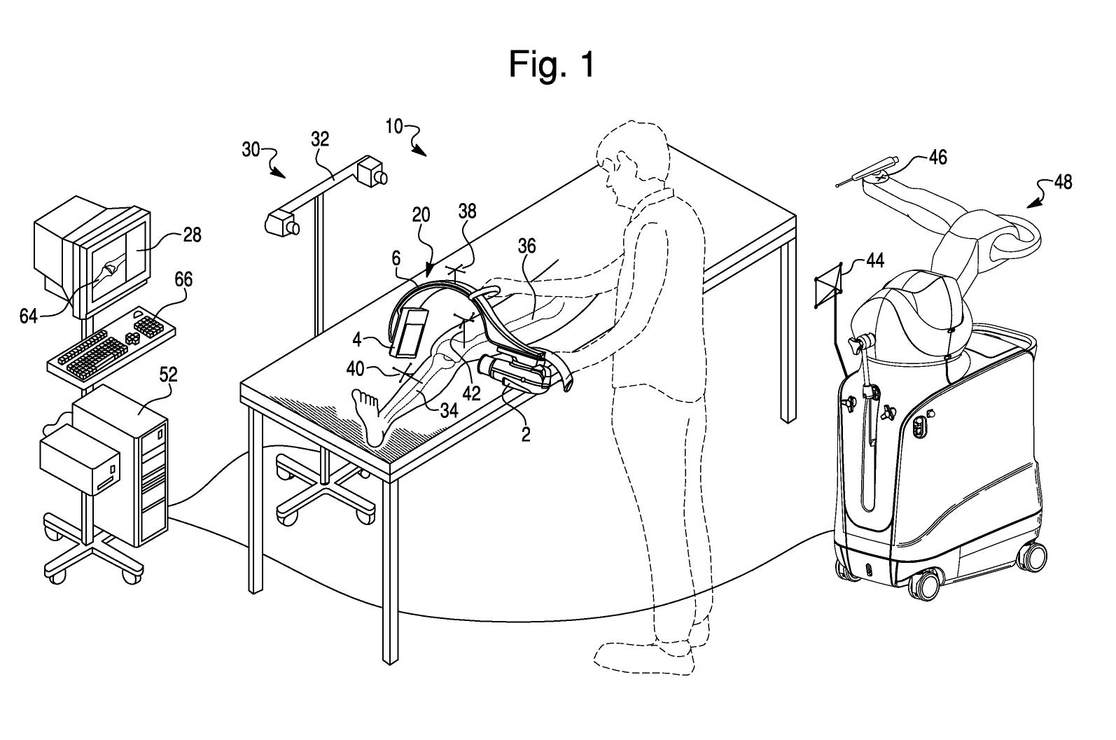

Referring to FIG. 1, according to an exemplary embodiment, an imaging system 10 includes a radiation source 2, a detector 4 fixed to the radiation source 2 such that the radiation source 2 and detector form a hand-held imaging device 20 configured to acquire image data, and a navigation system 30 configured to track the pose (i.e. position and orientation) of the hand-held imaging device 20.

Further referring to FIG. 1, the radiation source 2 of the imaging device 20 may be any portable radiation source. In a preferred embodiment, the radiation source 2 emits x-rays. Alternatively, the radiation source 2 may emit ultrasound, other types of electromagnetic radiation, or any other type of signal that can be detected by a detector to acquire a two-dimensional image of an object.

A detector 4 is fixed to the radiation source 2. The detector 4 may be a flat panel detector configured to be used with the radiation source 2. For example, the detector 4 may be an amorphous silicon (a-Si) thin-film transistor (TFT) detector or a cesium iodide (CsI) complementary metal-oxide semiconductor (CMOS) detector. In one embodiment, the detector 4 has dimensions of about five inches by six inches, although any size detector may be used. The detector 4 is configured to handle individual image acquisitions, in a mode known as “digital radiography.” Detector 4 may also be capable of a “continuous acquisition mode” to facilitate real-time, or near-real-time, continuous imaging.

The hand-held imaging device 20 is configured to acquire image data. The image data represents the radiation received by the detector 4, and the image data can be processed to form an image of an object placed between the radiation source 2 and the detector 4. The image data may be processed by a computer either local to (i.e. embedded within or directly connected to) the hand-held imaging device 20 or external to the hand-held imaging device 20. Similarly, the resulting image may be displayed on a local or an external display. The general process of utilizing the hand-held imaging device 20 to acquire image data is referred to herein as “image acquisition.”

The radiation source 2 and the detector 4 are preferably fixed to each other by a frame to form the hand-held imaging device 20. “Hand-held” means that a person of ordinary strength can freely carry and freely reposition the imaging device 20. Weight, mobility, and structure are at least three factors that may be considered to determine whether an imaging device is “hand-held” as defined herein. For example, without imposing specific weight limitations, the hand-held imaging device 20 preferably weighs sixteen pounds or less, more preferably fourteen pounds or less, more preferably twelve pounds or less, and most preferably ten pounds or less. However, depending on other factors, devices weighing more than sixteen pounds can also be considered hand-held if a person of ordinary strength is still able to freely carry and freely reposition the imaging device. The hand-held imaging device 20 may be connected to other components of the imaging system 10 by wires or other connections, but a user should be able to freely carry and freely reposition the imaging device 20 to capture images of an object placed between the radiation source 2 and the detector 4.

The frame 6 may be made out of any material suitable for fixing the radiation source 2 and detector 4 relative to each during imaging. The frame may be, for example, a lightweight carbon fiber frame. The frame 6 is rigid and curved in one embodiment, although the frame 6 may take other shapes that create a space for an object between the radiation source 2 and the detector 4. For example, the frame 6 may have sharp edges such that it forms half of a square, rectangle, or diamond. Alternatively, the frame 6 may be substantially circular, oval-shaped, square, or rectangular, with the radiation source 2 and detector 4 located on opposite sides of the enclosed shape. The frame 6 may form a continuous segment or loop (e.g. an oval with the radiation source 2 and image detector attached to the interior of the oval), or the frame 6 may include two or more portions separated, for example, by the radiation source 2 and detector 4.

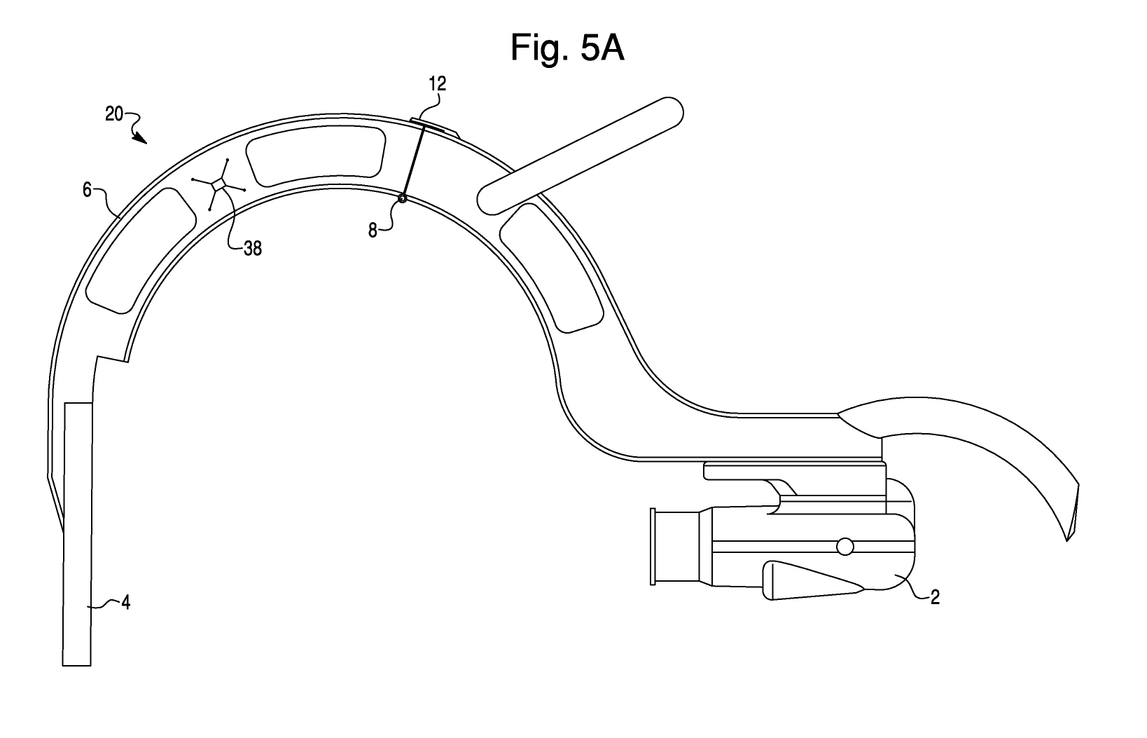

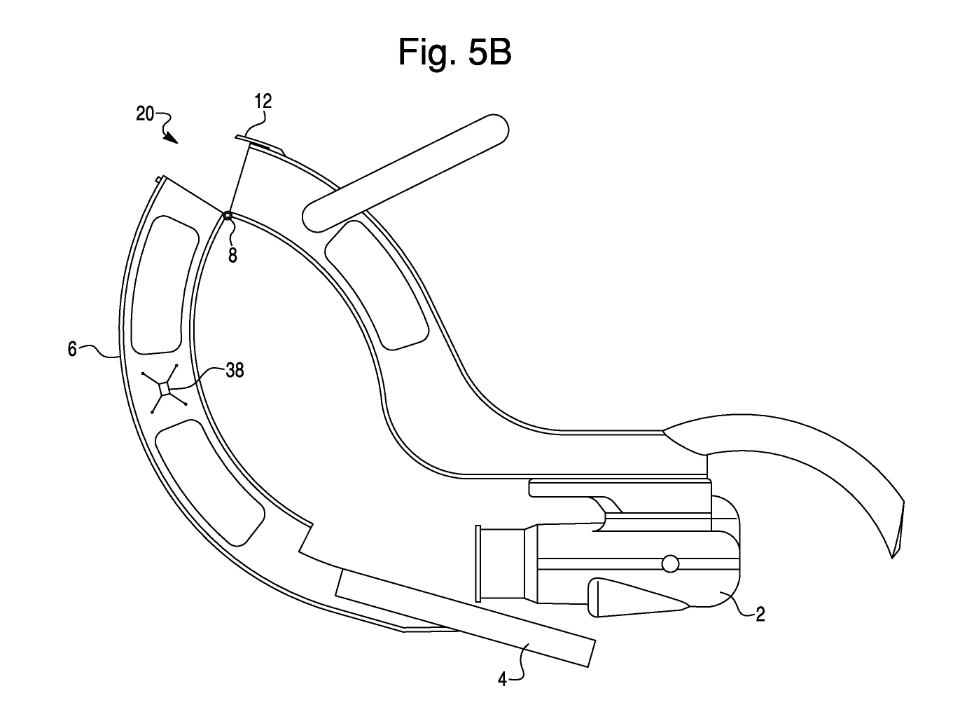

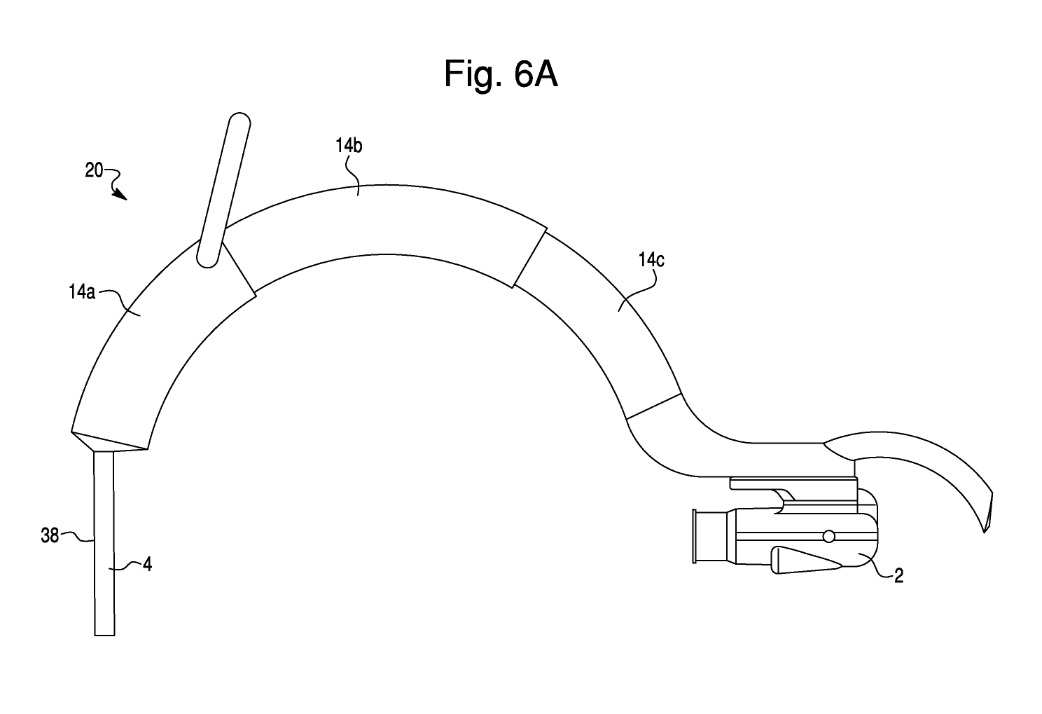

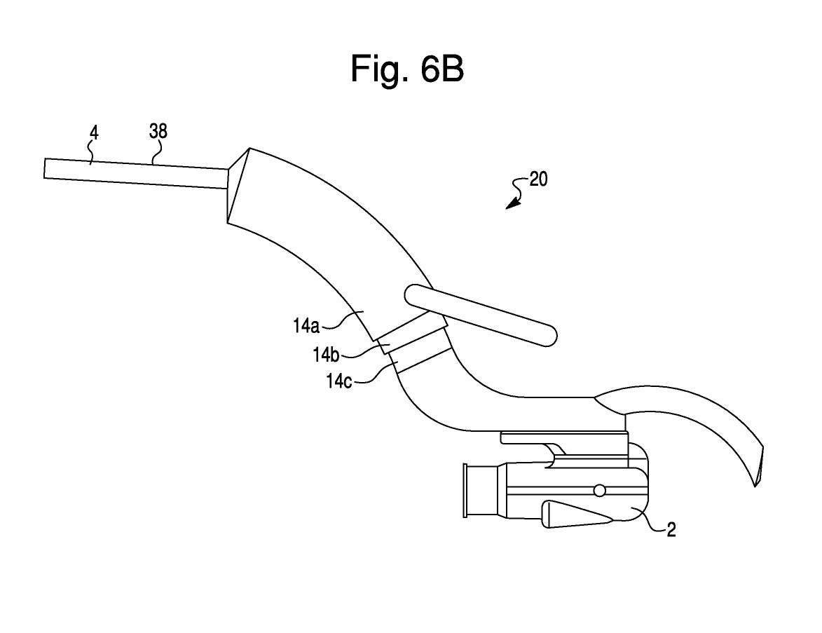

In one embodiment, the hand-held imaging device 20 is foldable or collapsible to further increase portability of the imaging device 20. For example, as shown in FIGS. 5A and 5B, the frame 6 may include one or more hinges 8, which lock in place with a lock 12 when the frame 6 is in an expanded state. A user can unlock the frame and fold the imaging device 20 into a collapsed state, as shown in FIG. 5B, when the imaging device 20 is not in use. Referring to FIG. 6A, the frame 6 may alternatively include segments 14a, 14b, 14c that are collapsible into each other. The segments may be held in an expanded state by mechanical locks, friction locks, twist locks, or any other known mechanism. FIG. 6B illustrates the embodiment of FIG. 6A with the imaging device 20 in a collapsed state.

The radiation source 2 and detector 4 may be adjustable relative to the frame 6. By adjusting the position of the frame segments and/or the radiation source 2 and detector 4, the imaging device 20 can be modified based on the size and shape of the object to be imaged. However, repositioning of the radiation source 2 relative to the detector 4 may require re-calibration of the imaging device 20 prior to additional imaging. In one method of calibration, a grid of radiopaque markers may be fixed to the front of the image detector in order to compute the intrinsic camera parameters, i.e. projection geometry, for each acquired image. This type of calibration, also known as “online” camera calibration, enables the radiation source and detector to be repositioned in real-time. The imaging device 20 may include mechanisms (e.g. mechanical locks, friction locks, etc.) to ensure stable positioning of the radiation source 2 and detector 4 relative to each other during image acquisition. As described below, the imaging device 20 is configured to be tracked by a navigation system, and in one embodiment, the radiation source 2 and detector 4 are tracked as a single unit (e.g. as part of the hand-held imaging device 20).

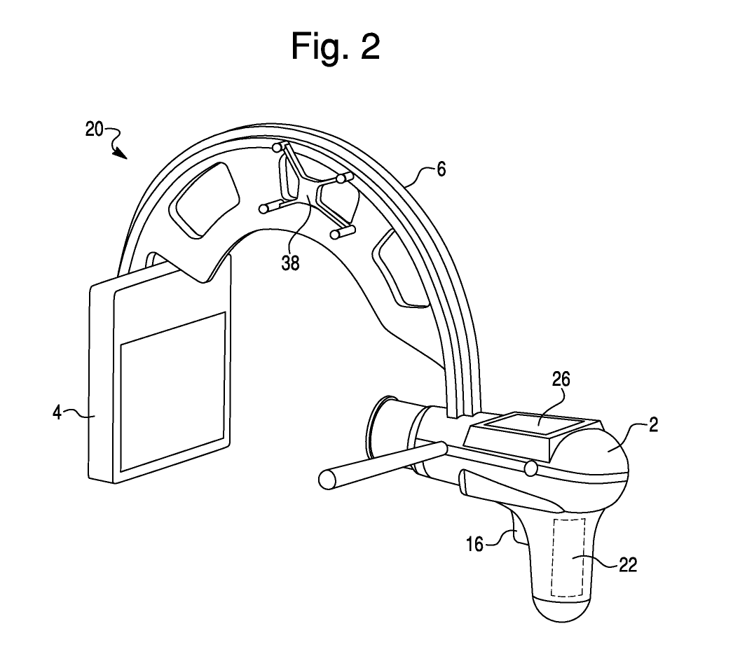

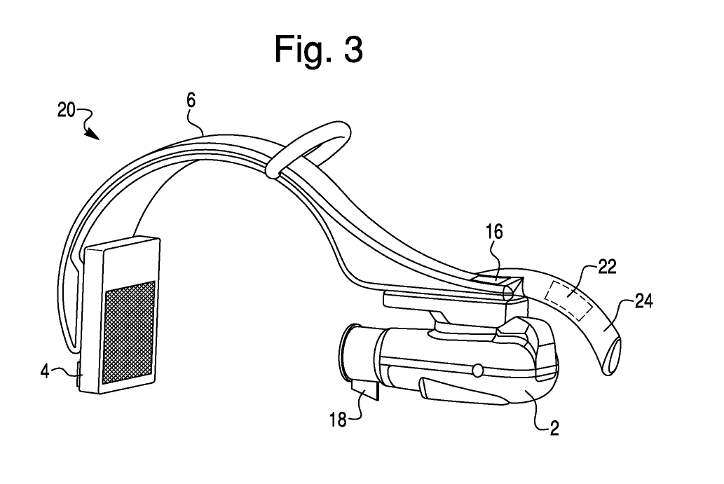

As shown in FIGS. 2 and 3, one embodiment of the hand-held imaging device 20 includes a trigger 16 to activate the radiation source 2 when a user desires to acquire an image using the hand-held imaging device 20. The trigger may be located at any location on the imaging device 20, such as on the handle as shown in FIG. 2 or on the frame 6 as shown in FIG. 3. The term “trigger” includes any type of mechanism configured to activate the radiation source 2 (e.g. push-button, switch, knob, etc.). The trigger may further take the form of a touch screen or virtual image that can be tapped or pressed to activate the radiation source 2. In an alternative embodiment, the “trigger” may include a mechanism that activates the radiation source 2 after receiving verbal commands from a user.

Referring to FIG. 3, the hand-held imaging device 20 may optionally include a laser 18 that emits a laser beam to assist a user in aligning the radiation source 2. The laser is mounted on or near the radiation source 2, although the laser could alternatively be mounted on or near the detector 4 or on any other suitable area of the imaging device 20. A user can use the laser as a guide to indicate the center of the resulting image. Based on viewing the laser beam on the object, the user can reposition the imaging device 20 to more accurately align the imaging device 20 to capture the desired image.

In one embodiment, the hand-held imaging device 20 is battery-operated. As used herein, “battery” includes one or more batteries as well as any other mobile source of power. The battery 22 is illustrated schematically in FIG. 2 incorporated into the radiation source 2 housing. Alternatively, the battery 22 may be housed in an extension of the frame (i.e. in a handle 24), as shown in FIG. 3. Preferably, the battery is rechargeable and can be removed from the imaging device 20 for ease of recharging and replacement. The imaging device 20 may also include an AC plug as an alternative source of power, an additional source of power, and/or to recharge the battery. One advantage of battery-operation is to eliminate the need for an electrical cable during use of the imaging device 20, which may hinder the imaging device's mobility. Furthermore, particularly when the imaging device 20 is being used for medical applications (e.g. for diagnosis, during a surgical procedure, during postoperative evaluation, etc.), eliminating additional cables increases safety in the physician's office or operating room.

The hand-held imaging device 20 may further include a display 26 (i.e. a local display 26). The local display 26 may be mounted on any portion of the hand-held imaging device 20, such as embedded within or on the radiation source 2, as shown in FIG. 2. The local display 26 may be configured as a touch screen and/or may include its own input device, thereby serving as a user interface and allowing a user to input information into the imaging device 20.

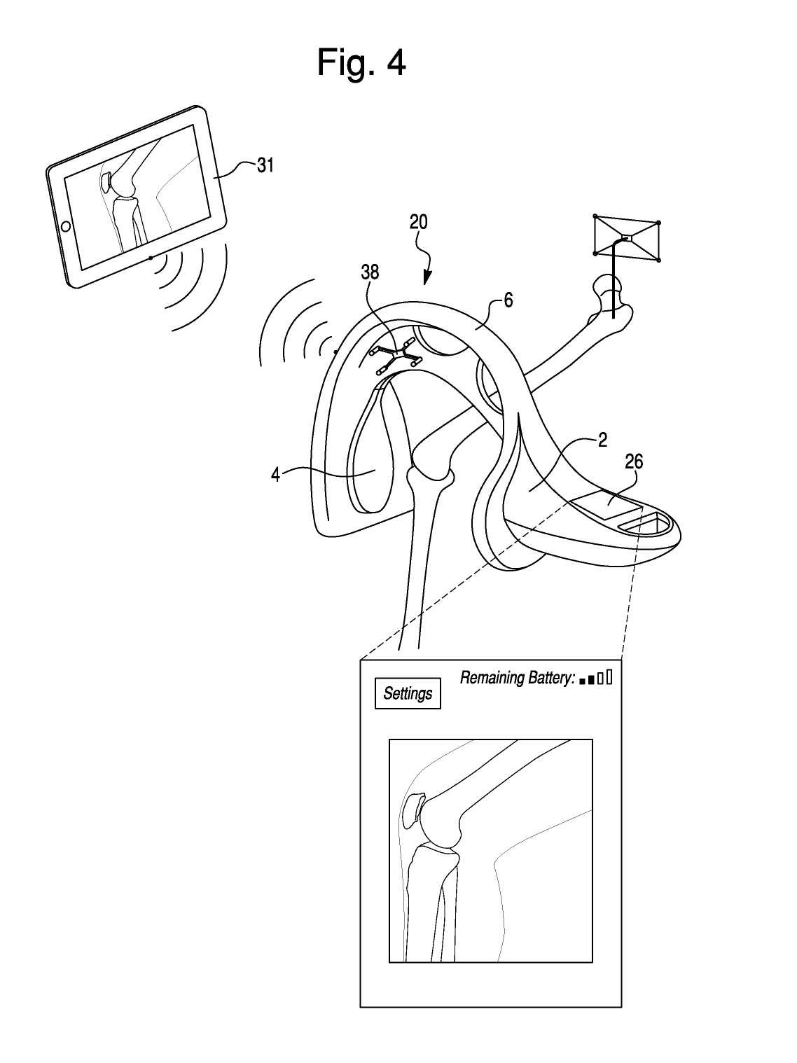

During use of the imaging device 20, the local display 26 may be configured to display an acquired image of an object and/or to display a predicted image of an object, as described below. Referring to FIG. 4, the local display 26 may further provide status information, such as remaining battery power and system errors, to a user of the imaging device 20. A user can quickly and easily modify various settings of the imaging device 20, such as exposure time, by using the touch screen or the input device of the local display 26.

Any features of the local display 26 described herein may also (or alternatively) be embodied by an external display, such as a display 28 of an imaging system 10, as shown in FIG. 1, or the display of a tablet computer 31 (e.g. iPad), as shown in FIG. 4. For example, a user could alter various settings of the imaging device 20 using the tablet computer 31. An external display 28, 30 may be configured to communicate with the hand-held imaging device 20 by either a wired or wireless connection. The ability of the imaging device 20 to perform numerous functions locally and to interact with external devices (e.g. external displays and computers) enhances the imaging device's convenience and applicability in a variety of situations.

The imaging system 10 further includes a navigation system 30 configured to track one or more objects to detect movement of the objects. The navigation system 30 includes a detection device 32 that obtains a pose of an object with respect to a coordinate frame of reference of the detection device 32. As the object moves in the coordinate frame of reference, the detection device 32 tracks the pose of the object to detect movement of the object. The navigation system 30 may be any type of navigation system 30 that enables the imaging system 10 to continually determine (or track) a pose of the hand-held imaging device 20 (or its components) as the imaging device 20 is being moved and repositioned by a user. For example, the navigation system 30 may be a non-mechanical tracking system, a mechanical tracking system, or any combination of non-mechanical and mechanical tracking systems. In a preferred embodiment, the navigation system 30 is configured to track the pose of the imaging device 20 in six degrees of freedom.

In one embodiment, the navigation system 30 includes a non-mechanical tracking system as shown in FIG. 1 and also as described in U.S. Pat. No. 8,010,180, titled “Haptic Guidance System and Method,” granted Aug. 30, 2011, and hereby incorporated by reference herein in its entirety. The non-mechanical tracking system is an optical tracking system that comprises a detection device 32 and a trackable element (or navigation marker 38) that is disposed on a tracked object and is detectable by the detection device 32. In one embodiment, the detection device 32 includes a visible light-based detector, such as a MicronTracker (Claron Technology Inc., Toronto, CN), that detects a pattern (e.g., a checkerboard pattern) on a tracking element. In another embodiment, the detection device 32 includes a stereo camera pair sensitive to infrared radiation and able to be positioned in an operating room where the surgical procedure will be performed. The marker is affixed to the tracked object in a secure and stable manner and includes an array of markers having a known geometric relationship to the tracked object. As is known, the markers may be active (e.g., light emitting diodes or LEDs) or passive (e.g., reflective spheres, a checkerboard pattern, etc.) and have a unique geometry (e.g., a unique geometric arrangement of the markers) or, in the case of active, wired markers, a unique firing pattern. In operation, the detection device 32 detects positions of the markers, and the imaging system 10 (e.g., the detection device 32 using embedded electronics) calculates a pose of the tracked object based on the markers' positions, unique geometry, and known geometric relationship to the tracked object. The tracking system 30 includes a marker for each object the user desires to track, such as the navigation marker 38 located on the hand-held imaging device 20.

The hand-held imaging device 20 may be utilized during a medical procedure performed with a haptically guided interactive robotic system, such as the haptic guidance system described in U.S. Pat. No. 8,010,180. For example, during use of the imaging device 20 for registration purposes (described below), the navigation system 30 may also include one or more anatomy markers 40, 42 (to track patient anatomy, such as a tibia 34 and a femur 36), a haptic device marker 44 (to track a global or gross position of the haptic device 48), and an end effector marker 46 (to track a distal end of the haptic device 48). In one embodiment, the hand-held imaging device 20 may be temporarily coupled to the distal end of the haptic device 48. The user can then interact with the haptically guided robotic system to acquire images. The haptic device 48 may assist image acquisition by guiding the hand-held imaging device to the proper location or by controlling the orientation of imaging device 20. In alternative uses of the imaging device 20 (e.g. postoperative assessment of implant component position, described below), the navigation system 30 might only include the navigation marker 38 on the imaging device 20 and one or more anatomy markers 40, 42.

As noted above, the navigation marker 38 is attached to the hand-held imaging device 20. FIG. 1 shows the navigation marker 38 attached to the exterior of the frame 6 of the imaging device 20, although the navigation marker 38 may be positioned in any suitable location to allow it to interact with the detection device 32. For example, in FIG. 2, the navigation marker 38 is located on a side of the frame 6. In FIGS. 6A and 6B, the navigation marker 38 is shown on the back of the image detector 4 of the imaging device 20. The navigation marker may be an optical array, for example, or an equivalent marker corresponding to the type of navigation system 30. During tracking of the imaging device 20, the poses of the radiation source 2 and detector 4 may be fixed relative to each other. This structure enables the navigation system 30 to track the imaging device 20 as a single, rigid unit. The radiation source 2 and detector 4 may be tracked separately, however, if the relationship between the radiation source 2 and the detector 4 is known during image acquisition.

Alternatively, a mechanical navigation system may be used to track the hand-held imaging device 20. For example, a mechanical linkage instrumented with angular joint encoders, such as the MicroScribe articulating arm coordinate measuring machine (AACMM) (GoMeasure3D, Newport News, VA), may be rigidly coupled to the hand-held imaging device 20 enabling the tracking system to continually determine (or track) a pose of the hand-held imaging device 20 as the imaging device 20 is being moved and repositioned by a user.

In one embodiment, the imaging system 10 further includes a cart to hold various components of the imaging system 10, such as computer 52. The cart may include a docking station for the hand-held imaging device 20. Docking the imaging device 20 can provide protection during transportation of the imaging device 20 and may also provide a convenient mechanism for charging the battery 22 of the imaging device 20.

During image acquisition, the hand-held imaging device 20 is synchronized with the navigation system 30. One method of synchronization includes placing an infrared LED on the frame of the imaging device 20. The infrared LED is programmed to emit light while an image is being acquired. The navigation system 30 senses the emitted light and uses the information to determine the pose of the imaging device 20 at the time the image is acquired. Synchronizing the images acquired by the hand-held imaging device 20 with the pose of the imaging device 20 ensures accurate determination of the pose of the acquired images.

Referring to FIG. 1, the imaging system 10 further includes a processing circuit, represented in the figures as a computer 52. The computer 52 is configured to communicate with the imaging device 20 and navigation system 30 and to perform various functions related to image processing, image display, registration, navigation, and image guidance. Functions described herein may be performed by components located either within the hand-held imaging device 20 (e.g. a circuit board) or external to the hand-held imaging device 20, as shown in FIG. 1. The hand-held imaging device may include a transmitter configured to wirelessly transmit data of any type to computer 52 located external to the imaging device 20. The hand-held imaging device 20 may further include memory to store software, image data, and corresponding acquired images for later retrieval or processing. In some embodiments, the computer 52 is a tablet computer 31 (see FIG. 4). The computer 52 or tablet computer 31 may receive the image data via the wireless connection from the hand-held imaging device 20, which advantageously reduces additional cables during use of the imaging device 20. The computer 52, alone or in combination with additional computers (e.g. located within haptic device 48) may be further adapted to enable the imaging system 10 to perform various functions related to surgical planning and haptic guidance.

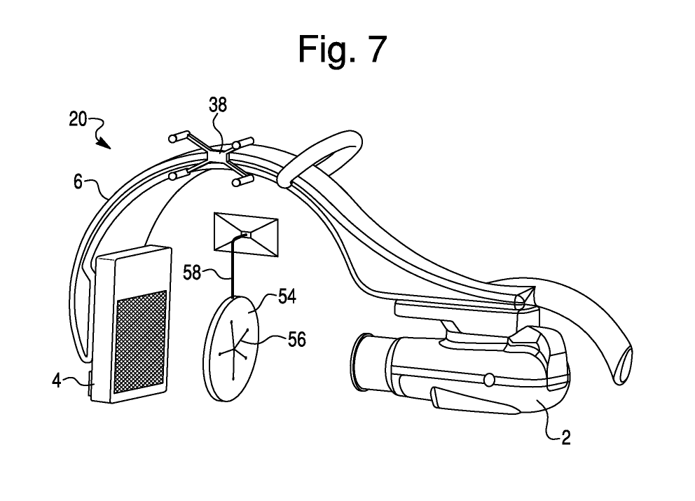

The hand-held imaging device 20 may be calibrated prior to use to determine the intrinsic parameters of the imaging device 20, including focal length and principal point. Referring to FIG. 7, calibration is performed using a calibration phantom 54 having a linear or radial pattern of radiopaque, fiducial markers 56 in a known, relative position. A calibration navigation marker 58 is placed on the calibration phantom 54 in a known, fixed location relative to the fiducial markers. Multiple images of the calibration phantom are acquired with the hand-held imaging device 20, and the corresponding coordinates of the calibration navigation marker 58 are determined for each image. Traditional camera calibration methods may then be used to solve for the best-fit focal length and principal point given the image coordinates of each of the fiducial markers. In addition, the recorded position of navigation marker 38 may be utilized, along with the estimated extrinsic camera parameters (determined from camera calibration), to determine the transformation between navigation marker 38 and the coordinate system of detector 4 (i.e. the “camera” coordinate system).

The hand-held imaging device may be utilized for bone registration. A method of bone registration according to one embodiment includes providing a three-dimensional representation of an anatomy of a patient; providing a hand-held imaging device 20 with a hand-held frame 6, a radiation source 2 fixed to the frame 6, and a detector 4 fixed to the frame 6; acquiring a two-dimensional image of the anatomy using the imaging device 20; tracking a pose of the imaging device 20 with a navigation system 30; and registering the two-dimensional image with the three-dimensional representation.

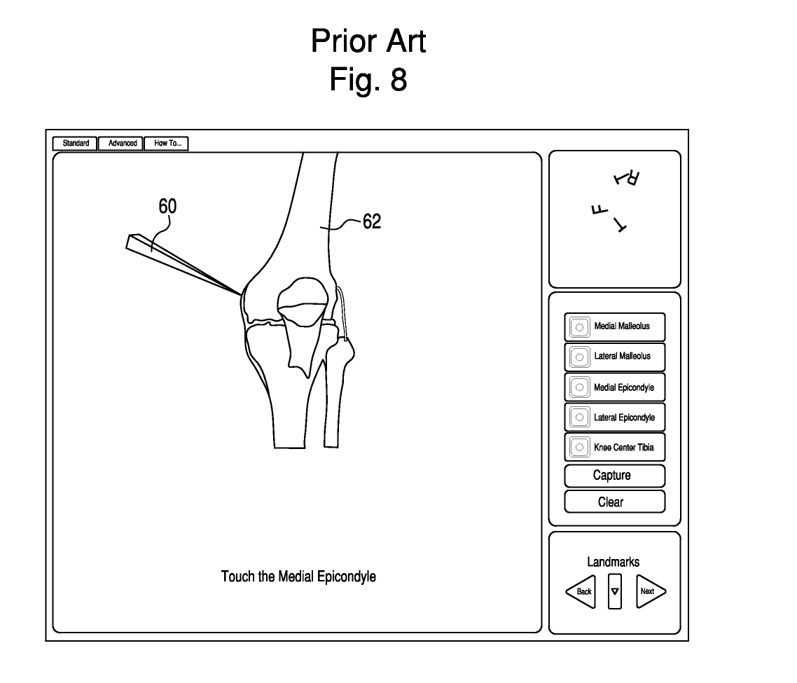

Registration is the process of correlating two coordinate systems, for example, by using a coordinate transformation process. FIG. 8 illustrates a display screen instructing a user employing a point-based method of registration during a computer-assisted surgery. Point-based registration methods utilize a tracked registration probe (represented virtually in FIG. 8 as virtual probe 60). The probe can be used to register a physical object to a virtual representation of the object by touching a tip of the probe to relevant portions of the object. For example, the probe may be used to register a femur of a patient to a three-dimensional virtual representation 62 of the femur by touching points on a surface of the femur.

There are challenges associated with utilizing point-based registration methods during computer-assisted surgeries. First, a surgeon must typically contact numerous points on the patient's bone to obtain an accurate registration. This process can be time-consuming. Second, the bone itself must be registered to the three-dimensional representation of the bone obtained prior to surgery, but the bone is often covered by 2-5 mm of cartilage. The surgeon must therefore push the probe through the cartilage to contact the bone. If the probe does not make it through the cartilage and does not contact the bone, the registration will not be as accurate. Inaccuracies may also result if the probe penetrates too far into the bone. Third, subjectivities may arise during implementation of point-based registration methods. Although the surgeon is typically guided by a display screen to point the registration probe to various anatomical landmarks, the surgeon decides exactly where to place the probe.

The method of bone registration according to one exemplary embodiment includes utilizing the hand-held imaging device 20 in connection with pose data from the navigation system 30 to register (i.e., map or associate) coordinates in one space to those in another space. In the embodiment shown in FIG. 1, the anatomy of a patient 34, 36 (in physical space) is registered to a three-dimensional representation of the anatomy (such as an image 64 in image space) by performing 2D/3D registration using one or more two-dimensional images captured by the hand-held imaging device 20. Based on registration and tracking data, the computer 52 of the imaging system 10 determines (a) a spatial relationship between the anatomy 34, 36 and the three-dimensional representation 64 and (b) a spatial relationship between the anatomy 34, 36 and the hand-held imaging device 20.

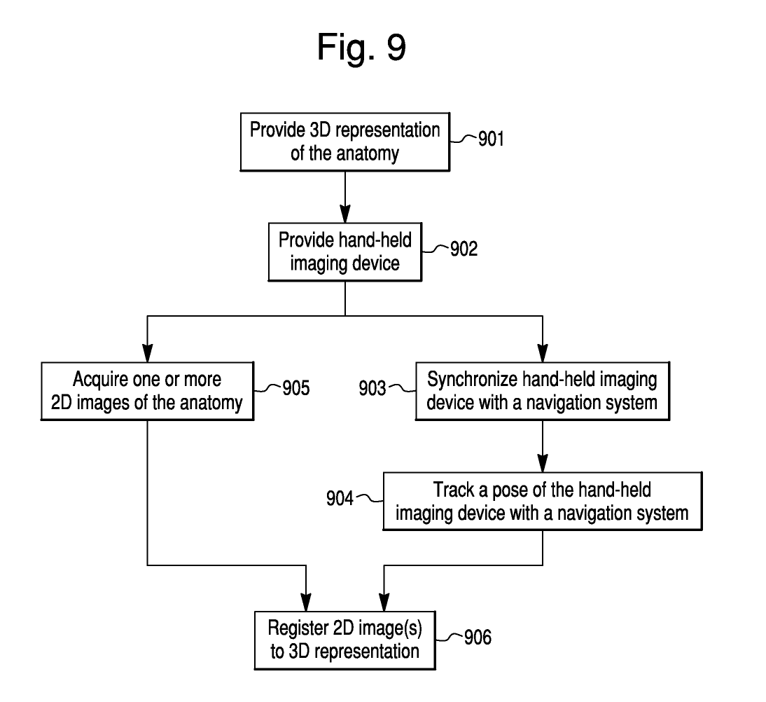

FIG. 9 is a flow chart illustrating use of the hand-held imaging device 20 for registration of a patient's anatomy prior to (or during) a surgical procedure. A three-dimensional representation 64 of some portion of the anatomy of a patient (referred to generally as “the anatomy”) is provided (step 901). The three-dimensional representation of the patient's anatomy may be obtained through a computed tomography (CT) scan, MRI, ultrasound, or other appropriate imaging technique. Alternatively, the three-dimensional representation may be obtained by selecting a three-dimensional model from a database or library of bone models. The user may use input device 66 to select an appropriate model, and the processor 52 may be programmed to select an appropriate model based on images and/or other information provided about the patient. The selected bone model can then be deformed based on specific patient characteristics, creating a three-dimensional representation of the patient's anatomy. The three-dimensional representation 64 may represent any portion of the anatomy suitable to be imaged by the hand-held imaging device 20, for example, a patient's knee or shoulder.

A hand-held imaging device 20 is also provided (step 902). The imaging device is synchronized with the navigation system 30 (step 903), and the navigation system 30 tracks a pose of the imaging device 20 (step 904). During use of the hand-held imaging device 20, the user places the imaging device 20 around the selected anatomy and activates the radiation source 2 to acquire one or more two-dimensional images of the anatomy (step 905). The image data is then processed, either within the imaging device 20 or by an external computer, and converted into a two-dimensional image. The two-dimensional image is displayed on the local display 26 and/or on an external display 28, and the user can confirm whether to keep or reject each image by interacting with a touch screen (e.g. display 26) on the imaging device 20 or with the input device 66. The patient's physical anatomy 34, 36 is then registered to the three-dimensional representation 64 of the patient's anatomy by 2D/3D registration (step 906). The tracked position of the patient's anatomy may serve as the global or reference coordinate system, which enables the patient's anatomy to move between the acquisition of images by the hand-held imaging device 20.

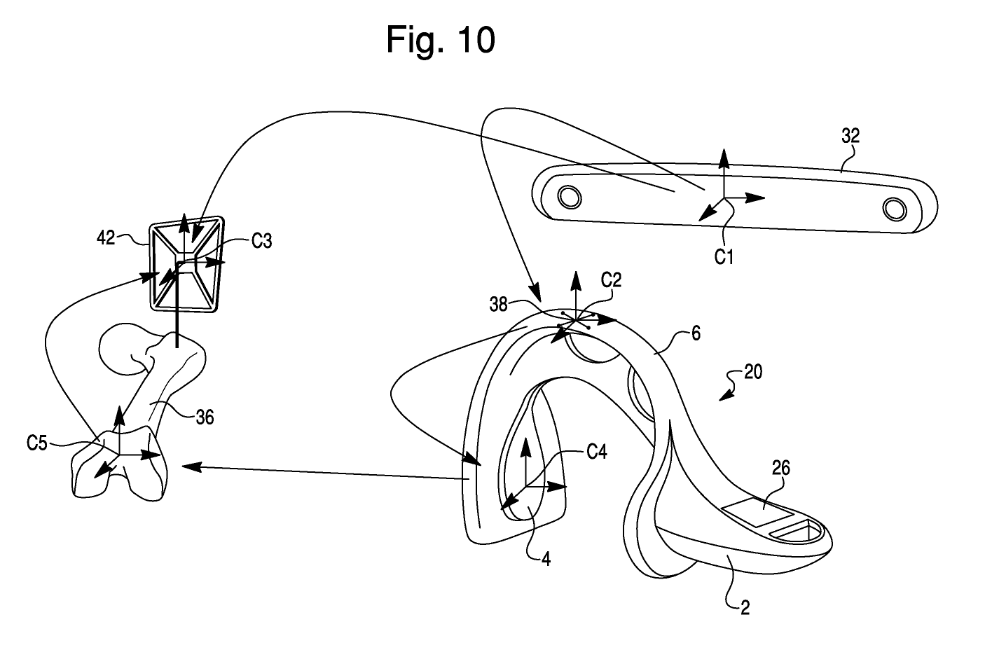

FIG. 10 is a diagram illustrating registration of the patient's anatomy (femur 36) and the relationships between various coordinate systems of the imaging system 10. The detection device 32 of the navigation system 30 tracks the imaging device 20 (having navigation marker 38) and the femur 36 (having an anatomy marker 42). The imaging system 10 therefore knows the spatial coordinates of coordinate system C2 of the navigation marker 38 and coordinate system C3 of the anatomy marker 42. The transformation between coordinate system C2 and coordinate system C4 of the imaging device 20 can be obtained by calibration of the imaging device (e.g. eye-in-hand calibration). After the imaging device 20 is utilized to acquire images of the femur 36, 2D/3D registration is performed to determine the transformation between the imaging device 20 (coordinate system C4) and the femur 36 (coordinate system C5). Once 2D/3D registration is performed, the coordinate transformation between coordinate system C5 and coordinate system C3 can be calculated. The imaging system 10 can then accurately track the femur 36 and provide image guidance via the three-dimensional representation shown on a display.

Registration of the patient's anatomy to a three-dimensional representation of the anatomy only requires a single two-dimensional image of the anatomy. In one embodiment, registration of a patient's knee is accomplished using a single lateral image of the patient's knee. However, registration accuracy may increase with additional two-dimensional images. Therefore, in another embodiment, at least two substantially orthogonal images of the anatomy are captured by the imaging device 20 for registration purposes.

Registration using the hand-held imaging device 20 during a surgical procedure overcomes certain challenges associated with point-based registration methods. Image acquisition and registration using the imaging device 20 is intended to be faster than the relatively more tedious point-based methods. Furthermore, 2D/3D registration using images captured by the imaging device 20 can be more accurate, as the surgeon does not have to ensure contact between a probe and bone.

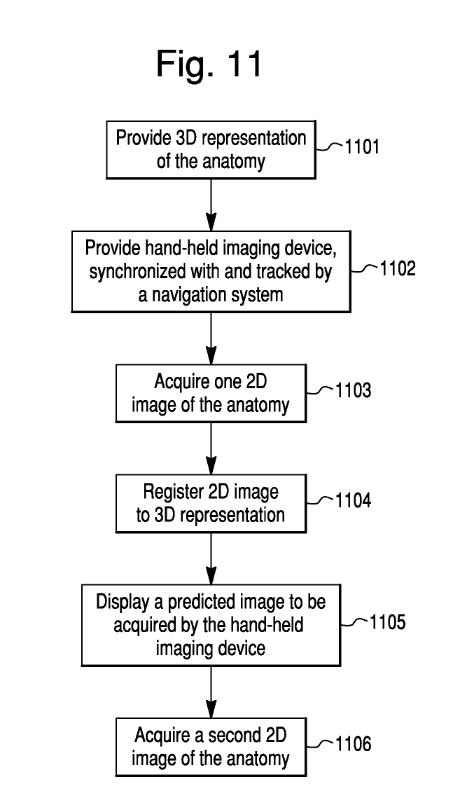

FIG. 11 is a flow chart illustrating a method of using the hand-held imaging device 20 for displaying a predicted image. In this embodiment, the imaging device 20 allows a user to view a predicted image prior to image acquisition. First, a three-dimensional representation 64 of an anatomy of a patient is provided, as described above (step 1101). Further, a hand-held imaging device 20 is provided (step 1102). The imaging device 20 is synchronized with and tracked by the navigation system 30. The imaging device 20 is then utilized to acquire at least one two-dimensional image of the anatomy (step 1103). After a first two-dimensional image is captured by the imaging device 20, registration may be performed between the two-dimensional image and the three-dimensional image 64 (step 1104). The navigation system 30 is then able to track the pose of both the imaging device 20 and the patient's anatomy. Information obtained in real-time by the tracking system (i.e. the pose of both the imaging device 20 and the patient's anatomy) allows the imaging system 10 to anticipate what an acquired image would look like as the imaging device 20 and the anatomy move during the procedure. The local display 26 and/or external display 28 then displays a real-time predicted image stream as the user repositions the imaging device or the patient moves (step 1105). The ability to display a predicted image(s) allows the user to view, in real-time and during repositioning of the hand-held imaging device 20, a relatively accurate prediction of the next captured image. A display of the predicted image(s) also assists the user in accurately aligning the hand-held imaging device 20 prior to acquiring subsequent images of the patient's anatomy. Increasing the accuracy of image alignment can reduce the number of images a user must take to capture the desired images. Because each image acquisition exposes the patient to additional radiation, reducing the number of acquired images advantageously minimizes the patient's radiation exposure. When the desired image appears on the display 26, 28, the user activates the radiation source 2 to acquire a second two-dimensional image (step 1106). The second (and subsequent) two-dimensional images can also be registered with the three-dimensional representation to improve the accuracy of the registration of the patient's anatomy. In another embodiment, the hand-held imaging device 20 can display a predicted image by emitting a low radiation dose as the user repositions the hand-held imaging device 20. In this embodiment, a predicted image can be displayed even prior to acquisition and registration of a first two-dimensional image of the anatomy (i.e. prior to step 1103).

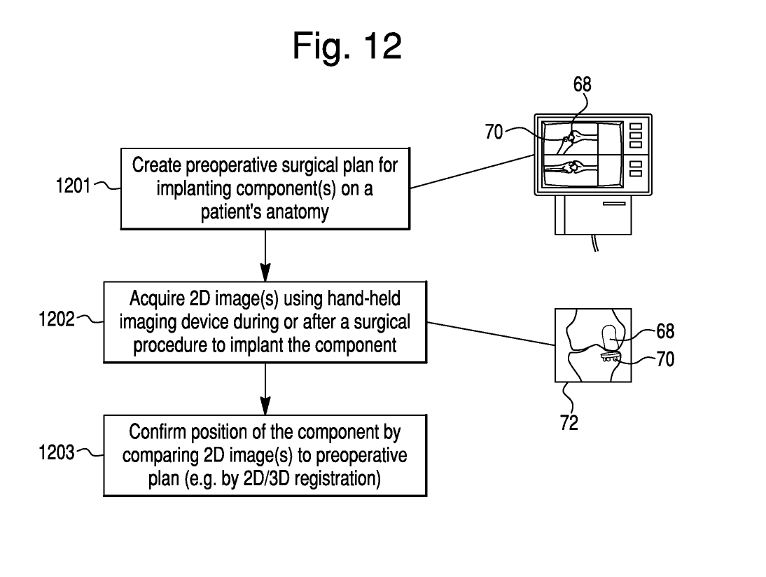

Referring to FIG. 12, a method for intraoperative or postoperative assessment using a hand-held imaging device 20 may include confirming a position of an implant component 68 during or after a surgical procedure. In this embodiment, the surgical procedure includes implanting one or more components 68, 70 into a patient according to a preoperative plan. A three-dimensional representation of the patient's anatomy is obtained prior to surgery, and a preoperative plan is established based on the three-dimensional representation.

As one illustration of intraoperative or postoperative assessment, a knee surgery may include establishing a preoperative plan (step 1201) for implanting and securing a tibial component 70 to a prepared surface of the tibia, as shown in FIG. 12. To confirm the position of the implanted component 70, the hand-held imaging device 20 is used to capture one or more two-dimensional images 72 of the patient's anatomy during the surgical procedure and/or after the completion of the surgical procedure to implant the component 70 (step 1202). The images should include both the implant (i.e. tibial component) and the bone (i.e. tibia), although the implant and bone may be in separate images if the bone is tracked during image acquisition. 2D/3D registration can then be performed on both the implant and the bone to determine their respective positions or poses relative to the preoperative plan (step 1203). This comparison can be done during the surgical procedure or shortly thereafter to confirm that the component 70 is in the desired position or pose. This comparison can additionally or alternatively be done at a later time to confirm that the component 70 has remained stationary over time.

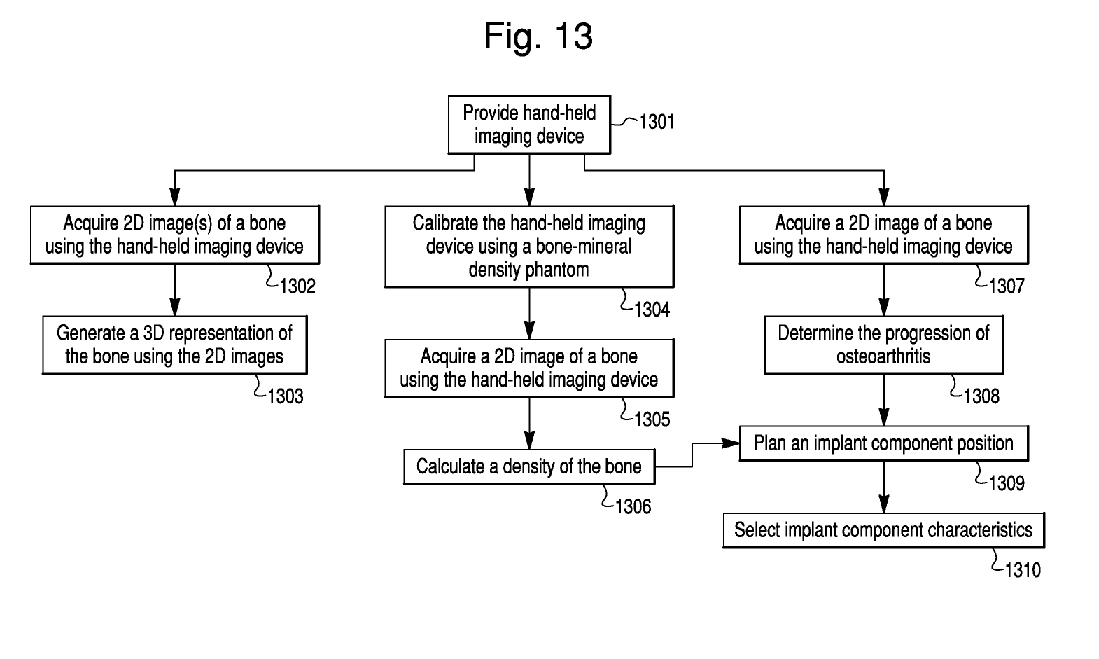

The hand-held imaging 20 can also be used for generating three-dimensional representations of a bone or any other object. As shown in FIG. 13, for example, a method of bone reconstruction includes providing a hand-held imaging device 20 (step 1301), acquiring one or more two-dimensional images of the bone using the hand-held imaging device 20 (step 1302), and generating a three-dimensional representation of the bone using one or more two-dimensional images (step 1303). In one embodiment, a three-dimensional model is selected from a database or library of bone models (as described above) comprised of data obtained from other patients, for example, through CT scans or other imaging techniques. By combining the selected model and one or more two-dimensional images of the patient's bone, a three-dimensional representation of the patient's bone can be constructed. Methods of bone reconstruction utilizing the imaging device 20 and/or the resulting reconstructed bone representation can be utilized in connection with any of the devices, systems, and methods described herein requiring a three-dimensional representation of a patient's anatomy.

The hand-held imaging device 20 is further intended to be useful in diagnosing a variety of medical diseases. For example, the hand-held imaging device 20 may be utilized to determine the bone density of a patient, which can be evaluated by a physician to diagnose osteoporosis. Referring again to FIG. 13, one embodiment of a method for determining bone density includes providing a hand-held imaging device 20 (step 1301), calibrating the imaging device 20 using a bone-mineral density phantom (step 1304), acquiring a two-dimensional image of a bone using the imaging device 20 (step 1305), and calculating a density of the bone (step 1306).

Calibration of the imaging device 20 according to step 1304 may include providing a bone-mineral density phantom, which represents human bone and contains a known amount of calcium. The imaging device 20 is calibrated by taking an image or images of at least one bone-mineral density phantom such that an unknown bone density can later be calculated. Calibration of the imaging device 20 for purposes of calculating bone density can be done separately or in connection with calibration for purposes of determining intrinsic parameters of the imaging device 20 as described above. In one embodiment, the calibration phantom 54 (FIG. 7) includes multiple inserts of different concentrations of calcium or an equivalent compound, allowing the phantom 54 to also serve as a bone-mineral density phantom. Performing both types of calibration simultaneously, or close in time to each other, minimizes the number of steps a user must perform prior to enabling all features of the imaging device 20, thereby increasing the efficiency and usefulness of the imaging device 20.

After calibration, the imaging device 20 is used to capture a two-dimensional image of a bone for which a density measurement is desired (step 1305). A computer located local or external to the imaging device 20 performs the necessary image analysis to calculate the density of the imaged bone. The imaging device 20 may be configured to output to the user on a local and/or external display, or by audio, the resulting bone density calculation(s). Alternatively, a physician may be able to estimate the density of the bone simply by viewing a display or printout of the radiographic image of the bone.

The hand-held imaging device 20 may further be utilized for diagnosing osteoarthritis. A method according to one embodiment (see FIG. 13) includes providing a hand-held imaging device 20 (step 1301), acquiring a two-dimensional image of a bone using the imaging device 20 (step 1307), and determining the progression of osteoarthritis (step 1308). In use, the physician places the hand-held imaging device 20 around the relevant portion of the patient's anatomy and activates the radiation source 2 to acquire at least one two-dimensional image. The physician can then determine the progression of osteoarthritis by viewing and analyzing the acquired two-dimensional image on a display local and/or external to the imaging device 20. The physician can evaluate the two-dimensional images to diagnose any disease and/or abnormality that is viewable on a radiographic image.

Utilizing the hand-held imaging device 20 for diagnostic purposes, including determination of bone density and diagnosis of osteoarthritis, may allow for more effective planning of a subsequent surgical procedure. For example, due to the mobility and ease of use of the hand-held imaging device 20, the patient can be imaged in the physician's office rather than having to travel to a separate imaging location. An additional trip to an external imaging location is therefore eliminated, reducing the time a patient must wait to receive an accurate diagnosis. Furthermore, images acquired in the physician's office can be evaluated to plan an implant component position and/or to select implant characteristics. Implant characteristics include, for example, implant type and implant size. Both planning an implant position and selecting implant characteristics can be based on a variety of factors, including bone density, progression of osteoarthritis, and other parameters of the patient's anatomy (e.g. width of the femur or tibia).

Embodiments of the present invention provide imaging systems, devices, and methods that provide numerous advantages over the prior art. For example, the present invention allows for faster, more convenient, and more efficient bone registration during surgical procedures, confirmation of surgical procedure results, diagnosis of various diseases, and surgical procedure planning

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish any connection steps, processing steps, comparison steps, and decision steps.

What is claimed is:

1. An imaging system, comprising:
a radiation source;
a detector fixed to the radiation source such that the radiation source and detector form a hand-held imaging device, wherein the hand-held imaging device is configured to acquire image data; and
a navigation system configured to track a pose of the hand-held imaging device.

2. The system of claim 1, further comprising a processing circuit configured to:
receive the image data, wherein the image data is of an anatomy of a patient; and
register the image data with a three-dimensional representation of the anatomy.

3. The system of claim 2, wherein the hand-held imaging device comprises a transmitter configured to wirelessly transmit the image data.

4. The system of claim 1, wherein the detector is fixed to the radiation source by a rigid frame.

5. The system of claim 1, wherein the detector is a flat-panel complementary metal-oxide semiconductor detector.

6. The system of claim 1, wherein the navigation system is an optical tracking system.

7. The system of claim 1, wherein the navigation system is configured to track the pose of the hand-held imaging device in six degrees of freedom.

8. The system of claim 1, wherein the hand-held imaging device is movable in six degrees of freedom.

9. The system of claim 1, wherein the hand-held imaging device further comprises a trigger to activate the radiation source.

10. The system of claim 1, wherein the hand-held imaging device comprises a laser configured to assist a user in aligning the radiation source.

11. The system of claim 1, wherein the hand-held imaging device is battery-operated.

12. The system of claim 1, wherein the hand-held imaging device further comprises a display.

13. The system of claim 1, wherein the hand-held imaging device weighs less than 16 pounds.

14. The system of claim 1, wherein the hand-held imaging device weighs less than 10 pounds.

15. The system of claim 1, wherein the hand-held imaging device is foldable.

16. A hand-held imaging device, comprising:
a hand-held frame;
a radiation source fixed to the frame; and
a detector fixed to the frame;
wherein the hand-held imaging device is configured to be tracked by a navigation system.

17. The imaging device of claim 16, wherein the frame is curved to create a space for an object between the radiation source and the detector.

18. The imaging device of claim 16, wherein the detector is a flat-panel complementary metal-oxide semiconductor detector.

19. The imaging device of claim 16, further comprising a navigation marker coupled to the frame.

20. The imaging device of claim 19, wherein the navigation marker is an optical array of an optical tracking system.

21. The imaging device of claim 16, further comprising a processing circuit configured to synchronize the hand-held imaging device with the navigation system.

22. The imaging device of claim 16, further comprising a transmitter configured to wirelessly transmit image data to a processing circuit.

23. The system of claim 16, wherein the hand-held frame is movable in six degrees of freedom.

24. The imaging device of claim 16, further comprising a trigger to activate the radiation source.

25. The imaging device of claim 16, further comprising a laser coupled to the frame and configured to assist a user in aligning the radiation source.

26. The imaging device of claim 16, wherein the device is battery-operated.

27. The imaging device of claim 16, further comprising a display coupled to the frame.

28. The imaging device of claim 16, wherein the device weighs less than 16 pounds.

29. The imaging device of claim 16, wherein the device weighs less than 10 pounds.

30. The imaging device of claim 16, wherein the device is foldable.

31. A method for bone registration using a hand-held imaging device, comprising:
providing a three-dimensional representation of an anatomy of a patient;
providing a hand-held imaging device, comprising:
a hand-held frame,
a radiation source fixed to the frame, and
a detector fixed to the frame;
acquiring a two-dimensional image of the anatomy using the imaging device;
tracking a pose of the imaging device with a navigation system; and
registering the two-dimensional image with the three-dimensional representation.

32. The method of claim 31, wherein the three-dimensional representation is of at least one of a human knee and a human shoulder.

33. The method of claim 31, further comprising:
acquiring a second two-dimensional image using the imaging device.

34. The method of claim 33, further comprising:
registering the second two-dimensional image with the three-dimensional representation.

35. The method of claim 33, further comprising:
displaying a predicted image of the second two-dimensional image on a display of the imaging device prior to acquiring the second two-dimensional image.

36. The method of claim 31, further comprising:
confirming a position of an implanted component.

37. The method of claim 36, wherein the step of confirming the position of an implanted component includes comparing the two-dimensional image to a preoperative plan.

38. The method of claim 37, wherein the two-dimensional image is acquired during a surgical procedure to implant the component.
